(19) United States
(12) Patent Application Publication
(10) Pub. No.: US 2016/0093807 A1
(43) Pub. Date: Mar. 31, 2016

(54) FULLERENE DERIVATIVE AND N-TYPE SEMICONDUCTOR MATERIAL

(71) Applicants: DAIKIN INDUSTRIES, LTD., Osaka (JP); OSAKA UNIVERSITY, Osaka (JP)

(72) Inventors: Takabumi NAGAI, Osaka (JP); Kenji ADACHI, Osaka (JP); Yoshio ASO, Osaka (JP); Yutaka IE, Osaka (JP); Makoto KARAKAWA, Osaka (JP)

(73) Assignees: OSAKA UNIVERSITY, Osaka (JP); DAIKIN INDUSTRIES, LTD., Osaka (JP)

(21) Appl. No.: 14/891,419

(22) PCT Filed: May 16, 2014
(86) PCT No.: PCT/JP2014/063127

§ 371 Date: Nov. 16, 2015

(30) Foreign Application Priority Data
May 16, 2013 (JP) 2013-104472
Oct. 2, 2013 (JP) 2013-207724

(51) Int. Cl. H01L 51/00; C07D 417/04; C07D 409/04; C07D 209/70

(52) U.S. Cl. CPC H01L 51/0047; C07D 209/70; C07D 417/04; C07D 409/04; H01L 51/4253

(57) ABSTRACT

The present invention is a material that exhibits excellent properties as an n-type semiconductor, in particular for use in organic thin-film solar cells. The present invention relates to a fullerene derivative represented by formula (1):

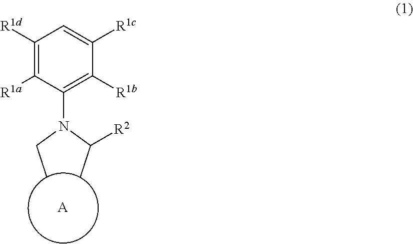

wherein
R^{1a} and R^{1b} are the same or different, and each represents a hydrogen atom or a fluorine atom;
R^{1c} and R^{1d} are the same or different, and each represents a hydrogen atom, a fluorine atom, alkyl, alkoxy, ester, or cyano;
R² represents (1) phenyl optionally substituted with at least one substituent selected from the group consisting of fluorine, alkyl, alkoxy, ester, and cyano, or (2) a 5-membered heteroaryl group optionally substituted with 1 to 3 methyl groups; and
ring A represents a fullerene ring.

### TECHNICAL FIELD

The present invention relates to a fullerene derivative, an n-type semiconductor material, and the like.

### BACKGROUND ART

Organic thin-film solar cells are formed by a coating technique with a solution of an organic compound, which is a photoelectric conversion material. The cells have various advantages: for example, 1) device production cost is low; 2) area expansion is easy; 3) the cells are more flexible than inorganic materials, such as silicon, thus enabling a wider range of applications; and 4) resource depletion is less likely. As such, organic thin-film solar cells have been developed, and the use of the bulk heterojunction structure has particularly led to a significant increase in conversion efficiency, thus attracting widespread attention.

For p-type semiconductor of the photoelectric conversion basic materials used for organic thin-film solar cells, poly-3-hexylthiophene (P3HT) is particularly known as an organic p-type semiconductor material exhibiting excellent performance. With an aim to obtain advanced materials, recent developments have provided compounds (donor-acceptor type n-conjugated polymers) that can absorb broad wavelengths of solar light or that have tuned energy levels, leading to significant improvements in the performance. Examples of such compounds include poly-p-phenylenevinylene and poly[[4,8-bis[(2-ethylhexyl)oxy]benzo[1,2-b:4,5-b′]dithiophene-2,6-diyl][3-fluoro-2-[(2-ethylhexyl)carbonyl]thieno[3,4-b]thiophenediyl]] (PTB7).

For n-type semiconductors as well, fullerene derivatives have been intensively studied, and [6,6]-phenyl-C₆₁-butyric acid methyl ester (PCBM) has been reported as a material having excellent photoelectric conversion performance (see the below-listed Patent Documents 1, 2, etc.). Nonetheless, there have been few reports that demonstrate stable and excellent conversion efficiency of fullerene derivatives except for PCBM.

Although fullerene derivatives for organic solar cells other than PCBM have been reported, the reports concern a comparison using special devices from which a power collection material of the positive electrode (ITO electrode) is removed (Non-patent Document 1), or fullerene derivatives only showing performance almost equivalent to that of PCBM (Non-patent Document 2). Although the disubstituted derivatives reported by Y.

Li et al. (Non-patent Document 3), when used with P3TH, achieved higher conversion efficiency than PCBM as reported by E. T. Hoke et al., the disubstituted derivatives exhibited only low conversion efficiency when used with a donor-acceptor π-conjugated polymer (Non-patent Document 4).

Thus, except for PCBM, advanced n-type materials capable of achieving high conversion efficiency, independently of p-type materials, have been unknown.

Several methods for synthesizing fullerene derivatives have been proposed. Methods known to be excellent from the standpoint of yield and purity include a method for synthesizing, using a diazo compound, a fullerene derivative having a 3-membered ring moiety and a method for synthesizing a fullerene derivative having a 5-membered ring moiety to which an azomethine ylide generated from a glycine derivative and an aldehyde is added.

The aforementioned PCBM is a fullerene derivative having a 3-membered ring moiety, and PCBM can be obtained by preparing a mixture of three types of products each having a fullerene backbone to which a carbene intermediate is added, and subjecting the mixture to a conversion reaction by light irradiation or heat treatment. However, the derivative having a 3-membered ring moiety obtained by this production method is restricted in terms of the introduction site of substituent and the number of substituents; thus, the development of novel n-type semiconductors has significant limitations.

Fullerene derivatives having a 5-membered ring moiety, on the other hand, are considered to be excellent because of their diverse structures. However, there have been few reports on the fullerene derivatives having excellent performance as an n-type semiconductor material for organic thin-film solar cells. One of a few examples is the fullerene derivative disclosed in the below-listed Patent Document 3.

### CITATION LIST

### Patent Documents

Patent Document 1: JP2009-084264A
Patent Document 2: JP2010-092964A
Patent Document 3: JP2012-089538A

### Non-Patent Documents

Non-patent Document 1: T. Itoh et al., Journal of Materials Chemistry, 2010, vol. 20, page 9,226
Non-patent Document 2: T. Ohno et al., Tetrahedron, 2010, vol. 66, page 7,316
Non-patent Document 3: Y. Li et al., Journal of American Chemical Society, 2010, vol. 132, page 1,377
Non-patent Document 4: E. T. Hoke et al., Advanced Energy Materials, 2013, vol. 3, page 220

### SUMMARY OF INVENTION

### Technical Problem

The present invention has been completed in view of the above-described status quo of the related art, and the major object is to provide a material having excellent performance as an n-type semiconductor, more specifically as an n-type semiconductor for photoelectric conversion elements such as organic thin-film solar cells.

### Solution to Problem

Patent Document 3 states that the fullerene derivative disclosed in the document shows high photoelectric conversion efficiency. A study conducted by the present inventors suggested that the basicity of the amine in the pyrrolidine moiety contained in the fullerene derivative is the major factor in this high photoelectric conversion efficiency.

A further study by the present inventors led to the following new findings: the sterically bulky structure of the substituent at position 2 of the pyrrolidine moiety affects the performance of the fullerene derivative as an n-type semiconductor; more specifically, a derivative having a more bulky substituent exhibits decreased conversion efficiency. However, the study also revealed that when the pyrrolidine moiety is not substituted at position 2, the fullerene derivative shows low solubility, and low conversion efficiency.

The present inventors conducted extensive research on the basis of these findings, and found that a fullerene derivative represented by formula (1) below has excellent performance as an n-type semiconductor.

The present invention provides a fullerene derivative represented by formula (1) below, and an n-type semiconductor material and the like consisting of the fullerene derivative.

### Item 1

A fullerene derivative represented by formula (1):

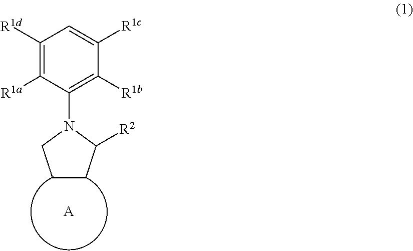

wherein
R¹a and R^{1b} are the same or different, and each represents a hydrogen atom or a fluorine atom;
R^{1c} and R^{1d} are the same or different, and each represents a hydrogen atom, a fluorine atom, alkyl optionally substituted with at least one fluorine atom, alkoxy optionally substituted with at least one fluorine atom, ester, or cyano;
R² represents
   (1) phenyl optionally substituted with at least one substituent selected from the group consisting of fluorine, alkyl, alkoxy, ester, and cyano,
   (2) a 5-membered heteroaryl group optionally substituted with 1 to 3 methyl groups, or
   (3) alkyl, alkoxy, ether, acyl, ester, or cyano; and
ring A represents a fullerene ring;
with the proviso that when R^{1a}, R^{1b}, R^{1c}, and R^{1d} are each a hydrogen atom, R² represents phenyl substituted with 1 or 2 fluorine atoms or a 5-membered heteroaryl group optionally substituted with 1 to 3 methyl groups.

### Item 2

The fullerene derivative according to Item 1, wherein
R^{1a} and R^{1b} are the same or different, and each represents a hydrogen atom or a fluorine atom;
at least one of R^{1a} and R^{1b} is a fluorine atom; and
R² is a group represented by the following formula:

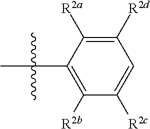

wherein,
R²a and R²b are the same or different, and each represents a hydrogen atom, a fluorine atom, alkyl, or alkoxy; and
R²c and R²d are the same or different, and each represents a hydrogen atom, a fluorine atom, alkyl, alkoxy, ester, or cyano.

### Item 3

The fullerene derivative according to Item 2, wherein
R^{1a} and R^{1b} arethe same or different, and each represents a hydrogen atom or a fluorine atom;
at least one of R^{1a} and R^{1b} is a fluorine atom;
R^{1c} and R^{1d} are the same or different, and each represents a hydrogen atom or a fluorine atom;
R²a and R²b are the same or different, and each represents a hydrogen atom, a fluorine atom, alkyl, or alkoxy; and
R²c and R²d each represents a hydrogen atom.

### Item 4

The fullerene derivative according to any one of Items 1 to 3, wherein the ring A is C₆₀ fullerene or C₇₀ fullerene.

### Item 5

An n-type semiconductor material consisting of the fullerene derivative according to any one of Items 1 to 4.

### Item 6

The n-type semiconductor material according to Item 5, which is for use in an organic thin-film solar cell.

### Item 7

An organic power-generating layer comprising the n-type semiconductor material according to Item 6.

### Item 8

A photoelectric conversion element comprising the organic power-generating layer according to Item 7.

### Item 9

The photoelectric conversion element according to Item 8, which is an organic thin-film solar cell.

### Item 10

The n-type semiconductor material according to Item 5, which is for use in a photosensor array.

### Item 11

The photoelectric conversion element according to Item 8, which is for use in a photosensor array.

### Advantageous Effects of Invention

The fullerene derivative according to the present invention is useful as an n-type semiconductor material, particularly an n-type semiconductor for photoelectric conversion elements such as organic thin-film solar cells.

### DESCRIPTION OF EMBODIMENTS

As used herein, “alkyl” refers to a linear or branched C₁₋₁₀ alkyl such as methyl, ethyl, propyl, isopropyl, butyl, isobutyl, sec-butyl, tert-butyl, pentyl, isopentyl, neopentyl, and hexyl, unless indicated otherwise.

As used herein, “alkoxy” refers to, for example, a group represented by RO— wherein R is alkyl, unless indicated otherwise.

As used herein, “ester” refers to, for example, a group represented by RCO₂— wherein R is alkyl, unless indicated otherwise.

As used herein, “ether” refers to a group having an ether bond (—O—), and includes a polyether group, unless indicated otherwise. The polyether group includes a group represented by formula: R^{a}—(O—R^{b})ₙ— wherein R^{a} is alkyl, R^{b} is the same or different in each occurrence, and is alkylene, and n is an integer of 1 or more. The alkylene is a divalent group formed by removing one hydrogen atom from the above-described alkyl).

As used herein, “acyl” includes alkanoyl, unless indicated otherwise. As used herein, “alkanoyl” refers to, for example, a group represented by RCO— wherein R is alkyl, unless indicated otherwise.

As used herein, a “5-membered heteroaryl group” refers to, for example, a 5-membered heteroaryl group containing as members of its ring at least one heteroatom (e.g., 1, 2, or 3 heteroatoms) selected from the group consisting of oxygen, sulfur, and nitrogen, unless indicated otherwise; examples of the 5-membered heteroaryl group include pyrrolyl (e.g., 1-pyrrolyl, 2-pyrrolyl, and 3-pyrrolyl), furil (e.g., 2-furil, and 3-furil), thienyl (e.g., 2-thienyl, and 3-thienyl), pyrazolyl (e.g., 1-pyrazolyl, 3-pyrazolyl, and 4-pyrazolyl), imidazolyl (e.g., 1-imidazolyl, 2-imidazolyl, and 4-imidazolyl), isoxazolyl (e.g., 3-isoxazolyl, 4-isoxazolyl, and 5-isoxazolyl), oxazolyl (e.g., 2-oxazolyl, 4-oxazolyl, and 5-oxazolyl), isothiazolyl (e.g., 3-isothiazolyl, 4-isothiazolyl, and 5-isothiazolyl), thiazolyl (e.g., 2-thiazolyl, 4-thiazolyl, and 5-thiazolyl), triazolyl (e.g., 1,2,3-triazole-4-yl, and 1,2,4-triazole-3-yl), oxadiazolyl (e.g., 1,2,4-oxadiazole-3-yl, and 1,2,4-oxadiazole-5-yl), and thiadiazolyl (e.g., 1,2,4-thiadiazole-3-yl, and 1,2,4-thiadiazole-5-yl).

The following describes in detail a fullerene derivative according to the present invention, an n-type semiconductor material, and the like consisting of).

### Fullerene Derivative

The fullerene derivative according to the present invention is represented by the following formula (1)

### Formula (1)

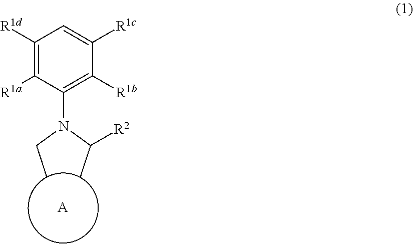

wherein
R^{1a} and R^{1b} are the same or different, and each represents a hydrogen atom or a fluorine atom;
R^{1c} and R^{1d} are the same or different, and each represents a hydrogen atom, a fluorine atom, alkyl optionally substituted with at least one fluorine atom, alkoxy optionally substituted with at least one fluorine atom, ester, or cyano;
R² represents
(1) phenyl optionally substituted with at least one substituent selected from the group consisting of fluorine, alkyl, alkoxy, ester, and cyano,
(2) a 5-membered heteroaryl group optionally substituted with 1 to 3 methyl groups, or
(3) alkyl, alkoxy, ether, acyl, ester, or cyano; and
ring A represents a fullerene ring;
with the proviso that when R^{1a}, R^{1b}, R^{1c}, and R^{1d} are each a hydrogen atom, R² represents phenyl substituted with 1 or 2 fluorine atoms or a 5-membered heteroaryl group optionally substituted with 1 to 3 methyl groups.

The fullerene derivative according to the present invention has a group represented by the following partial structural formula,

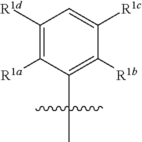

which is attached to the nitrogen atom, a constituent atom of the pyrrolidine moiety in formula (1), wherein the symbols are as defined above. This weakens the base property attributable to the nitrogen atom, thereby providing excellent properties as an n-type semiconductor material.

Preferable examples of the group include 2-fluorophenyl and 2,6-difluorophenyl.

R² is preferably a group represented by the following formula:

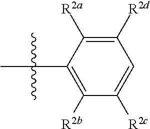

wherein R²a and R²b are the same or different, and each represents a hydrogen atom, a fluorine atom, alkyl, or alkoxy; and
R²c and R²d are the same or different, and each represents a hydrogen atom, a fluorine atom, alkyl, alkoxy, ester, or cyano.

Preferable examples of R² include 2-fluorophenyl, 2,6-difluorophenyl, 2-methoxyphenyl, and 2,6-dimethoxyphenyl.

In a preferable embodiment of the present invention, R^{1a} and R^{1b} are the same or different, and each represents a hydrogen atom or a fluorine atom; at least one of R^{1a} and R^{1b} is a fluorine atom; and R² is a group represented by the following formula:

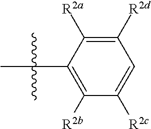

wherein,
R²a and R²b are the same or different, and each represents a hydrogen atom, a fluorine atom, alkyl, or alkoxy; and
R²c and R²d are the same or different, and each represents a hydrogen atom, a fluorine atom, alkyl, alkoxy, ester, or cyano.

In the embodiment, more preferably, R^{1a} and R^{1b} are the same or different, each represents a hydrogen atom or a fluorine atom, and at least one of R^{1a} and R^{1b} is a fluorine atom; R^{1c} and R^{1d}are the same or different, and each represents a hydrogen atom or a fluorine atom; R²a and R²b are the same or different, and each represents a hydrogen atom, a fluorine atom, alkyl, or alkoxy; and R²c and R²d are each a hydrogen atom.

Ring A is preferably C₆₀ fullerene or C₇₀ fullerene, and more preferably C₆₀ fullerene.

The fullerene derivative represented by formula (1) may be a mixture of a fullerene derivative having C₆₀ fullerene as ring A and a fullerene derivative having C₇₀ fullerene as ring A.

As used herein, C₆₀ fullerene may be represented by the following structural formula, which is often used in this technical field:

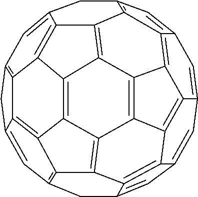 [SMILES: c1c2c3c4c(cc5c6c4c4c7c3c3c1Cc1cc8cc9cc%10cc(c%11c%10c%10c9c9c8c1c3c7c9c%10c4c%116)C5)C2]

When ring A is C₆₀ fullerene, the fullerene derivative of formula (1) can be represented by the following formula.

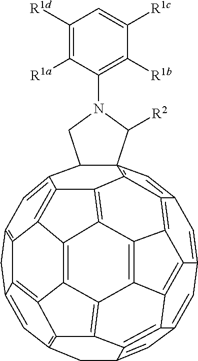

A fullerene derivative of one embodiment of the present invention is represented by the following formula (1A):

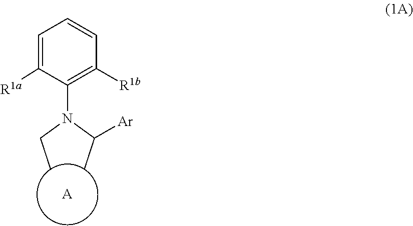

wherein,
R^{1a} and R^{1b} are the same or different, and each represents a hydrogen atom or a fluorine atom;
Ar is phenyl optionally substituted with 1 or 2 fluorine atoms, or a 5-membered heteroaryl group optionally substituted with 1 to 3 methyl groups; and
ring A represents a fullerene ring,
with the proviso that when R^{1a} and R^{1b} are both hydrogen atoms, Ar is phenyl substituted with 1 or 2 fluorine atoms, or a 5-membered heteroaryl group optionally substituted with 1 to 3 methyl groups.

The fullerene derivative in this embodiment has a compact, substituted or unsubstituted phenyl (i.e., phenyl, 2-fluorophenyl, or 2,6-difluorophenyl) represented by the following partial structural formula,

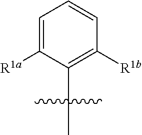

which is attached to the nitrogen atom, a constituent atom of the pyrrolidine moiety in formula (1A). This weakens the base property attributable to the nitrogen atom, thereby providing excellent properties as an n-type semiconductor material.

In this embodiment, Ar is preferably phenyl substituted with 1 or 2 fluorine atoms, or a 5-membered heteroaryl group optionally substituted with 1 to 3 methyl groups.

Because Ar is such a compact, substituted or unsubstituted aromatic group, the fullerene derivative of the present invention can exhibit excellent properties as an n-type semiconductor material.

In this embodiment, the “phenyl substituted with 1 or 2 fluorine atoms” represented by Ar is preferably phenyl substituted with 1 or 2 fluorine atoms at the ortho position (i.e., 2-fluorophenyl, or 2,6-difluorophenyl).

In this embodiment, preferable examples of Ar include phenyl, 2-fluorophenyl, 2,6-difluorophenyl, 2-thienyl, and 2-thiazolyl, and more preferable examples include phenyl, 2-fluorophenyl, and 2,6-difluorophenyl.

In a preferable embodiment of the fullerene derivative represented by formula (1A), at least one of R^{1a} and R^{1b} is a fluorine atom.

In another preferable embodiment of the fullerene derivative represented by formula (1A), R^{1a} and R^{1b} are both hydrogen atoms, and
Ar is phenyl substituted with 1 or 2 fluorine atoms, or a 5-membered heteroaryl group optionally substituted with 1 to 3 methyl groups.

Because the fullerene derivative represented by formula (1) shows excellent solubility in various organic solvents, it is easy to form a thin film using a coating technique.

In addition, the fullerene derivative represented by formula (1) easily forms a bulk heterojunction structure, when used as an n-type semiconductor material to prepare an organic power-generating layer, together with an organic p-type semiconductor material.

### Method for Producing a Fullerene Derivative

The fullerene derivative represented by formula (1) can be produced by a known method for producing a fullerene derivative, or by a method complying therewith.

Specifically, the fullerene derivative represented by formula (1) can be synthesized, for example, in accordance with the following scheme. The symbols indicated in the scheme are as defined above.

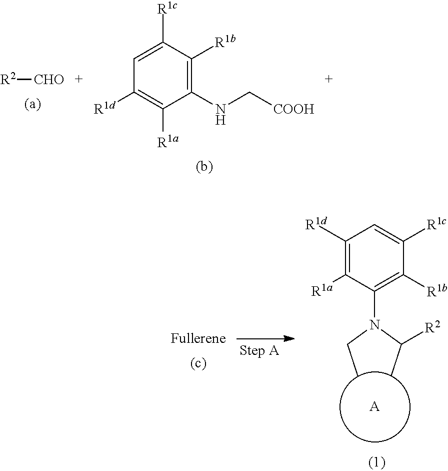

### Step A

In step A, a glycine derivative (compound (b)) reacts with an aldehyde compound (compound (a)) and a fullerene (compound (c)) to thereby obtain a fullerene derivative (compound (1)) represented by formula (1).

Although the amount ratio of the aldehyde compound (compound (a)), the glycine derivative (compound (b)), and the fullerene (compound (c)) is arbitrarily determined, the aldehyde compound (compound (a)) and the glycine derivative (compound (b)) are each typically added in an amount of 0.1 to 10 moles, and preferably 0.5 to 2 moles, per mole of the fullerene (compound (c)), from the standpoint of achieving high yield.

The reaction is carried out without a solvent or in a solvent. Examples of solvents include carbon disulfide, chloroform, dichloroethane, toluene, xylene, chlorobenzene, and dichlorobenzene. Of these, chloroform, toluene, chlorobenzene, and the like are preferable. These solvents may be mixed in suitable proportions.

The reaction temperature is typically within the range of room temperature to about 150° C., and preferably within the range of about 80 to about 120° C. As used herein, the room temperature is within the range of 15 to 30° C.

The reaction time is typically within the range of about 1 hour to about 4 days, and preferably within the range of about 10 to about 24 hours.

The obtained compound (1) can optionally be purified by a conventional purification method. For example, the obtained compound (1) can be purified by silica gel column chromatography (as a developing solvent, for example, hexane-chloroform, hexane-toluene, or hexane-carbon disulfide is preferably used), and further purified by HPLC (preparative GPC) (as a developing solvent, for example, chloroform or toluene is preferably used).

The aldehyde compound (compound (a)), the glycine derivative (compound (b)), and the fullerene (compound (c)) used in step A are all known compounds; the compounds can be synthesized by a known method or a method complying with a known method, and are also commercially available.

Specifically, the aldehyde compound (compound (a)) can be synthesized, for example, by the below-described method (a1), (a2), or (a3).

In the reaction formulae describing these methods, R² is as defined in formula (1), and corresponds to R² of the desired fullerene derivative.

### Method (a1): Oxidation of Alcohol Represented by R²—CH₂OH

For oxidation in this method, for example, the following known methods can be used: (i) a method using chromic acid, manganese oxide, or the like as an oxidant, (ii) swern oxidation using dimethylsulfoxide as an oxidant, or (iii) an oxidation method using hydrogen peroxide, oxygen, air, or the like in the presence of a catalyst.

### Method (a2): Reduction of Carboxylic Acid Represented by R²—COOH, Acid Halide Thereof, Ester Thereof, or Acid Amide Thereof

For reduction in this method, for example, the following known methods can be used: (i) a method using metal hydride as a reducing agent, (ii) a method comprising hydrogen reduction in the presence of a catalyst, or (iii) a method using hydrazine as a reducing agent.

### Method (a3): Carbonylation of Halide Represented by R²—X (X represents a halogen)

For carbonylation in this method, for example, a method comprising forming an anion from the halide using n-BuLi and introducing a carbonyl group thereinto can be used. As a carbonyl group-introducing reagent, amide compounds such as N,N-dimethylformamide (DMF); or N-formyl derivatives of piperidine, morpholine, piperazine, or pyrrolidine can be used.

Specifically, the glycine derivative (compound (b)) can be synthesized, for example, by the below-described method (b1), (b2), or (b3).

In the reaction formulae showing these methods, Ar¹ is a group represented by the following formula:

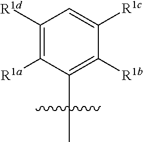

wherein the symbols are as defined above.

### Method (b1): Reaction Between Aniline Derivative and Halogenated Acetic Acid

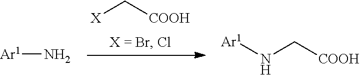

The reaction can employs water, methanol, ethanol, or a mixture thereof as a solvent, and can optionally be carried out as necessary in the presence of a base.

### Method (b2): Reaction Between Aniline Derivative and Halogenated Acetic Acid Ester, and Hydrolysis of Glycine Derivative Ester Obtained by Reaction

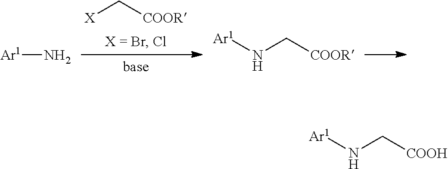

In this method, the reaction between an aniline derivative and a halogenated acetic acid ester can employs, for example, methanol or ethanol as a solvent, and can be carried out in the presence of a base such as acetate, carbonate, phosphate, and a tertiary amine. The hydrolysis of a glycine derivative ester can typically be carried out in the presence of a water-soluble alkali at room temperature.

### Method (b3): Reaction Between Aromatic Halide and Glycine

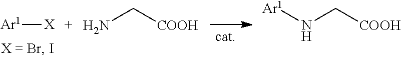

The reaction employs, for example, monovalent copper as a catalyst, and can be carried out in the presence of a bulky amine, an amino acid, or an amino alcohol. As a reaction solvent, water, methanol, ethanol, or a mixture thereof is preferably used. The reaction temperature is from room temperature to about 100° C.

As described above, the fullerene derivative according to the present invention can be synthesized by a simple method using a glycine derivative and an aldehyde derivative as starting materials; thus, the fullerene derivative can be produced at low cost.

### Use of Fullerene Derivative

The fullerene derivative according to the present invention can be suitably used as an n-type semiconductor material, particularly as an n-type semiconductor material for photoelectric conversion elements such as organic thin-film solar cells.

When used as an n-type semiconductor material, the fullerene derivative according to the present invention is typically used in combination with an organic p-type semiconductor material (organic p-type semiconductor compound).

Examples of organic p-type semiconductor materials include poly-3-hexylthiophene (P3HT), poly-p-phenylenevinylene, poly-alkoxy-p-phenylenevinylene, poly-9,9-dialkylfluorene, and poly-p-phenylenevinylene.

Because of the many approaches to use these materials in solar cells in the past and their ready availability, these materials can easily provide devices that exhibit stable performance.

To achieve higher conversion efficiency, donor-acceptor type n-conjugated polymers capable of absorbing long-wavelength light because of their narrowed bandgap (low bandgap) are effective.

These donor-acceptor n-conjugated polymers comprise donor units and acceptor units, which are alternately positioned.

Examples of usable donor units include benzodithiophene, dithienosilole, and N-alkyl carbazole, and examples of usable acceptor units include benzothiadiazole, thienothiophene, and thiophene pyrrole dione.

Specific examples include high-molecular compounds obtained by combining these units, such as poly(thieno[3,4-b]thiophene-co-benzo[1,2-b:4,5-b′]thiophene) (PTBx series), and poly(dithieno[1,2-b:4,5-b′][3,2-b:2′,3′-d]silole-alt-(2,1,3-benzothiadiazole).

Of these, the following are preferable:
(1) poly({4,8-bis[(2-ethylhexyl)oxy]benzo[1,2-b:4,5-b′]dithiophene-2,6-diyl}{3-fluoro-2-[(2-ethylhexyl)carbonyl]thieno[3,4-b]thiophenediyl}) (PTB7, the structural formula is shown below);
(2) poly[(4,8-di(2-ethylhexyloxy)benzo[1,2-b:4,5-b′]dithiophene)-2,6-diyl-alt-((5-octylthieno[3,4-c]pyrrol-4,6-dione)-1,3-diyl) (PBDTTPD, the structural formula is shown below);
(3) poly[(4,4′-bis(2-ethylhexyl)dithieno[3,2-b:2′,3′-d]silole)-2,6-diyl-alt-(2,1,3-benzothiadiazole)-4,7-diyl](PSBTBT, the structural formula is shown below);
(4) poly[N-9″-heptadecanyl-2,7-carbazole-alt-5,5-(4′,7′-di-2-thienyl-2′,1′,3′-benzothiadiazole)](PCDTBT, the structural formula is shown below); and
(5) poly[1-(6-{4,8-bis[(2-ethylhexyl)oxy]-6-methy1benzo[1,2-b:4,5-b′]dithiophene-2-yl}{3-fluoro-4-methylthieno[3,4-b]thiophene-2-yl}-1-octanone) (PBDTTT-CF, the structural formula is shown below)

Of these, more preferable examples include PTB-based compounds comprising as an acceptor unit thieno[3,4-b]thiophene having a fluorine atom at position 3, and yet more preferable examples include PBDTTT-CF and PTB7.

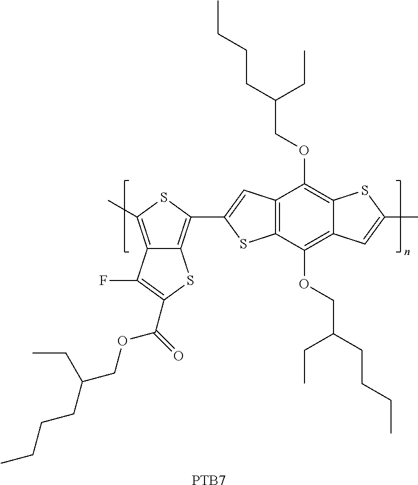

wherein n represents the number of repeating units.

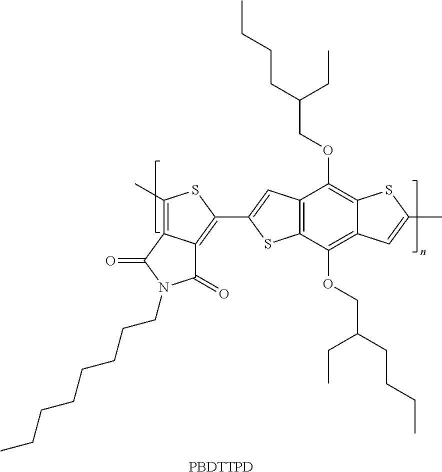

wherein n represents the number of repeating units.

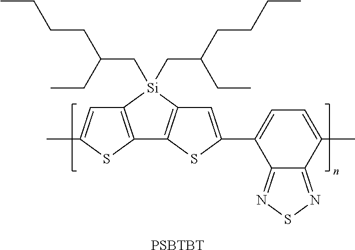

wherein n represents the number of repeating units.

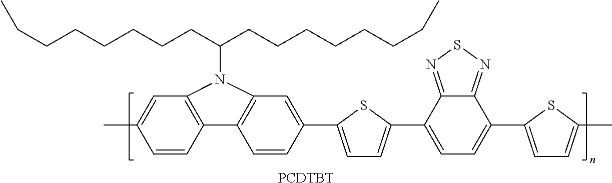

wherein n represents the number of repeating units.

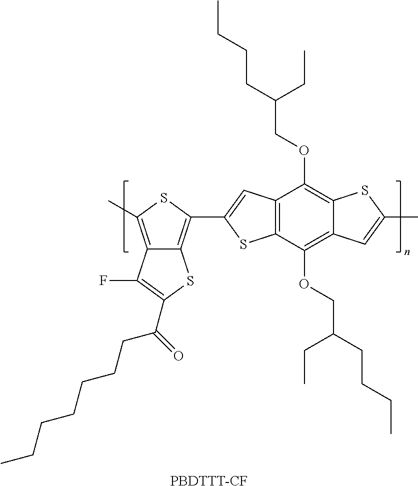

wherein n represents the number of repeating units.

An organic power-generating layer prepared by using the fullerene derivative according to the present invention as an n-type semiconductor material in combination with an organic p-type semiconductor material can achieve high conversion efficiency.

Because of its excellent solubility in various organic solvents, the fullerene derivative according to the present invention, when used as an n-type semiconductor material, enables the preparation of an organic power-generating layer by a coating technique, and also simplifies the preparation of an organic power-generating layer having a large area.

The fullerene derivative according to the present invention is a compound having excellent compatibility with organic p-type semiconductor materials as well as a suitable self-aggregating property. Thus, the fullerene derivative, when used as an n-type semiconductor material (organic n-type semiconductor material), can easily form an organic power-generating layer having a bulk junction structure. The use of such an organic power-generating layer enables the production of an organic thin-film solar cell or photosensor with high conversion efficiency.

Accordingly, the use of the fullerene derivative according to the present invention as an n-type semiconductor material enables the production of an organic thin-film solar cell having excellent performance at low cost.

An alternative application of the organic power-generating layer comprising (or consisting of) the n-type semiconductor material of the present invention is the use of the layer in an image sensor for digital cameras. In response to the demand for advanced functions (higher definition) in digital cameras, existing image sensors consisting of a silicon semiconductor are considered to suffer from lower sensitivity. Amid the demand, recent years have seen promises of achieving higher sensitivity and higher definition by using an image sensor consisting of an organic material with high photosensitivity. Materials for forming the light-receiving part of such a sensor need to absorb light with a high sensitivity and efficiently generate an electrical signal therefrom. In response to this demand, because of its ability to efficiently convert visible light into electrical energy, an organic power-generating layer comprising (or consisting of) the n-type semiconductor material of the present invention can have high performance as a material for the above-described light-receiving part of the sensor.

### -Type Semiconductor Material

The n-type semiconductor material according to the present invention consists of a fullerene derivative according to the present invention.

### Organic Power-Generating Layer

The organic power-generating layer according to the present invention comprises a fullerene derivative of the present invention as an n-type semiconductor material (n-type semiconductor compound).

The organic power-generating layer according to the present invention can be a light conversion layer (photoelectric conversion layer).

The organic power-generating layer according to the present invention typically comprises the aforementioned organic p-type semiconductor material (organic p-type semiconductor compound) in combination with the fullerene derivative according to the present invention, i.e., the n-type semiconductor material according to the present invention.

The organic power-generating layer according to the present invention typically consists of the n-type semiconductor material according to the present invention and the organic p-type semiconductor material.

The organic power-generating layer according to the present invention preferably has a bulk heterojunction structure formed by the n-type semiconductor material of the present invention and the organic p-type semiconductor material.

The organic power-generating layer according to the present invention is prepared, for example, by dissolving the n-type semiconductor material of the present invention and the aforementioned organic p-type semiconductor material in an organic solvent, and forming a thin film from the obtained solution on a substrate using a known thin-film forming technique, such as spin coating, casting, dipping, inkjet, and screen printing.

In formation of thin-film of an organic power-generating layer, the fullerene derivative according to the present invention has excellent compatibility with organic p-type semiconductor materials (preferably, P3HT, or PTB7) and suitable self-aggregating property. Therefore, it enables easy production of an organic power-generating layer comprising the fullerene derivative of the present invention, as an n-type semiconductor material, and an organic p-type semiconductor material, with the layer formed in a bulk heterojunction structure.

### Organic Thin-Film Solar Cell

The organic thin-film solar cell according to the present invention comprises the above-described organic power-generating layer of the present invention.

Thus, the organic thin-film solar cell of the present invention exhibits high conversion efficiency.

The structure of the organic thin-film solar cell is not particularly limited, and the organic thin-film solar cell may have the same structure as that of a known organic thin-film solar cell. The organic thin-film solar cell according to the present invention can also be produced in accordance with a known method for producing an organic thin-film solar cell.

One example of the organic thin-film solar cell comprising the fullerene derivative is a solar cell comprising, disposed on a substrate in series, a transparent electrode (negative electrode), a charge transport layer on the negative electrode side, an organic power-generating layer, a charge transport layer on the positive electrode side, and an opposite electrode (positive electrode). The organic power-generating layer is preferably a thin-film semiconductor layer (i.e., a photoelectric conversion layer) comprising an organic p-type semiconductor material and the fullerene derivative of the present invention as an n-type semiconductor material, with the layer formed in a bulk heterojunction structure.

In solar cells having the above-described structure, known materials can suitably be used as materials for layers other than the organic power-generating layer. Specific examples of electrode materials include aluminium, gold, silver, copper, and indium tin oxide (ITO). Examples of charge transport layer materials include PFN(poly[9,9-bis(3′-(N,N-dimethylamino)propyl-2,7-fluorene)-alt-2,7-(9,9-dioctylfluorene)]) and MoO₃ (molybdenum oxide).

### Photosensor

As described above, the photoelectric conversion layer obtained by the present invention can effectively function as an image sensor light-receiving part of advanced digital cameras. As compared with conventional photosensors including a silicon photodiode, a photosensor including the photoelectric conversion layer obtained by the present invention can receive an image in a well-lighted area without overexposure as well as a clear image in a poorly lighted area. This makes it possible to obtain an image with higher quality than those of conventional cameras. An photosensor comprises a silicon substrate, an electrode, a light-receiving part consisting of a photoelectric conversion layer, a color filter, and a microlens. The light-receiving part can be about several hundred nanometers in thickness, a fraction of the thickness of conventional silicon photodiodes.

### EXAMPLES

The following Examples describe the present invention in more detail. However, the present invention is not limited to the Examples.

The annotation of the symbols and abbreviations used in the Examples is shown below. In addition, symbols and abbreviations typically used in the technical field to which the present invention pertains may also be used throughout this specification.
s: singlet
d: doublet
d-d: double doublet p0 t: triplet
m: multiplet
Calcd: calculated value
Found: actual measured value

In the following Examples, GPC columns manufactured by Japan Analytical Industry Co., Ltd. were used (2 columns, 2H and 1H, of the Jaigel H Series were connected for use).

### Synthesis Example 1

### Synthesis of Compound 1

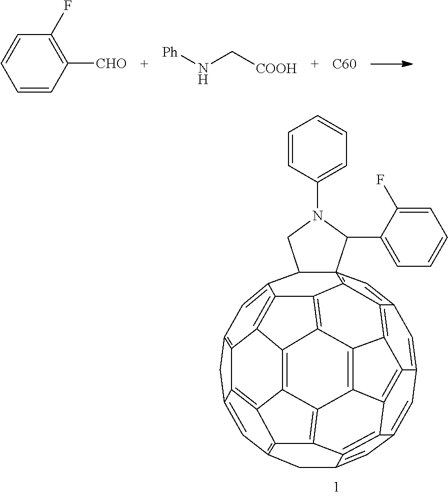

2-fluorobenzaldehyde (62 mg, 0.5 mmol), N-phenylglycine (151 mg, 1 mmol) and C₆₀ fullerene (350 mg, 0.5 mmol) were stirred in 100 mL of toluene at 120° C. for 15 hours. After cooling, the solvent was distilled off, and the reaction product was separated by column chromatography (SiO₂, n-hexane:toluene=20:1 to 5:1) to obtain Compound 1 (72.1 mg, yield: 15.4%). Compound 1 was further purified by preparative GPC (chloroform).
¹H-NMR (CDCl₃) δ: 5.09 (1H, d, J=9.9 Hz), 5.65 (1H, d, J=9.9 Hz), 6.61 (1H, s), 7.02-7.18 (3H, m), 7.20-7.28(2H, m), 7.28-7.42 (4H, m), 7.84 (1H, d-d, J=6.3, 6.3 Hz). ¹⁹F-NMR (CDCl₃) δ: −114.0-−115.5 (m).
MS (FAB) m/z 934 (M+1). HRMS calcd for C₇₄H4₁₃FN 934.1032; found 934.1023.

### Synthesis Example 2

### Synthesis of Compound 2

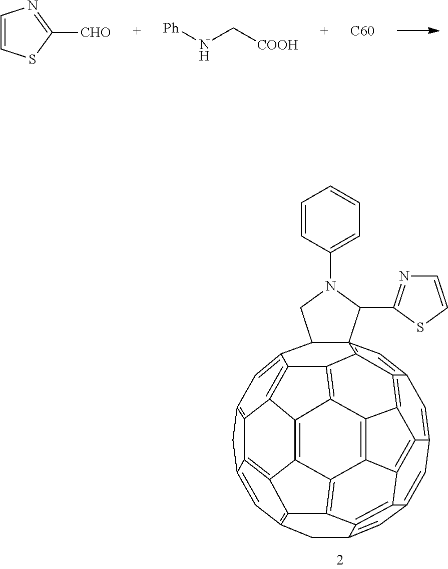

2-thiazole carbaldehyde (56 mg, 0.5 mmol), N-phenylglycine (76 mg, 0.5 mmol), and C₆₀ fullerene (175 mg, 0.25 mmol) were stirred in 100 mL of toluene at 120° C. for 62 hours. After cooling, the solvent was distilled off, and the reaction product was seprated by column chromatography (SiO₂, n-hexane:toluene=1:1 to toluene) to obtain Compound 2 (95 mg, yield: 41%). Compound 2 was further purified by preparative GPC (chloroform).
¹H-NMR (CDCl₃) δ: 5.27 (1H, d, J=9.9 Hz), 5.78 (1H, d, J=9.9 Hz), 6.91 (1H, s), 7.06 (1H, t, J=7.1 Hz), 7.30-7.46 (5H, m), 7.84 (1H, D, J=3.2 Hz).
MS (FAB) m/z 922 (M+). HRMS calcd for C₇₁H₁₀N₂S 922.0565; found 922.0562.

### Synthesis Example 3

### Synthesis of Compound 3

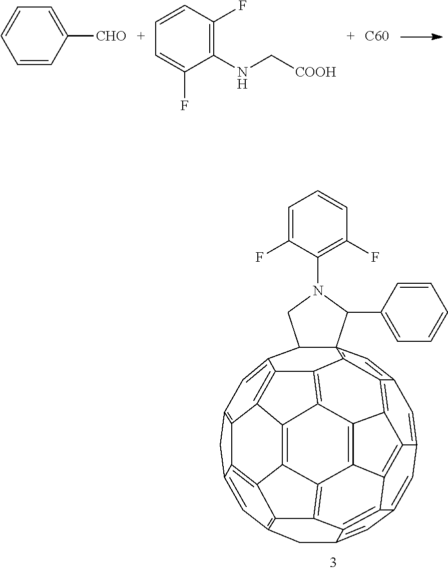

C₆₀ fullerene (360 mg, 0.5 mmol), benzaldehyde (212 mg, 2 mmol), and N-(2,6-difluorophenyl)glycine (187 mg, 1 mmol) were stirred in chlorobenzene (100 mL) at 130° C. for 4 days. After cooling, the solvent was distilled off, and the reaction product was separated by silica gel column chromatography (n-hexane:toluene=20:1 to 5:1) to obtain Compound 3 (108 mg, yield: 22.8%). Compound 3 was further purified by preparative GPC (chloroform).
¹H-NMR (CDCl₃) δ: 5.12 (1H, d, J=9.1 Hz), 5.26 (1H, d, J=9.1 Hz), 6.46 (1H, s), 6.96 (2H, t, J=8.7 Hz), 7.12-7.35 (4H, m), 7.77 (2H, d, J=7.5 Hz).
¹⁹F-NMR (CDCl₃) δ: −117.06-−117.15 (m).
MS (FAB) m/z 951 (M+). HRMS calcd for C₇₄H₁₁F₂N 951.0860; found 951.0861.

### Synthesis Example 4

### Synthesis of Compound 4

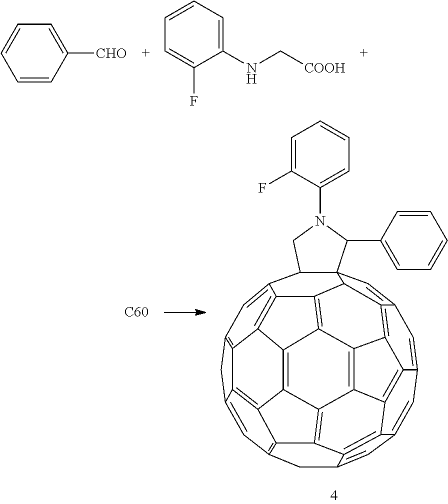

Fullerene C₆₀ (360 mg, 0.5 mmol), benzaldehyde (106 mg, 1 mmol), and N-(2-fluorophenyl)glycine (169 mg, 1 mmol) were stirred in chlorobenzene (100 mL) at 130° C. for 4 days. After cooling, the solvent was distilled off, and the reaction product was separated by silica gel column chromatography (n-hexane:toluene=20:1 to 5:1) to obtain Compound 4 (177 mg, yield: 37.9%). Compound 4 was further purified by preparative GPC (chloroform).
¹H-NMR (CDCl₃) δ: 4.74 (1H, d, J=9.6 Hz), 5.66 (1H, d, J=9.6 Hz), 6.10 (1H, s), 7.10-7.38 (7H, m), 7.77 (2H, d, J=7.3 Hz).
¹⁹F-NMR (CDCl₃) δ: −119.50-−119.75 (m).
MS (FAB) m/z 934 (M+1). HRMS calcd for C₇₄H₁₃FN 934.1032; found 934.1052.

### Synthesis Example 5

### Synthesis of Compound 5

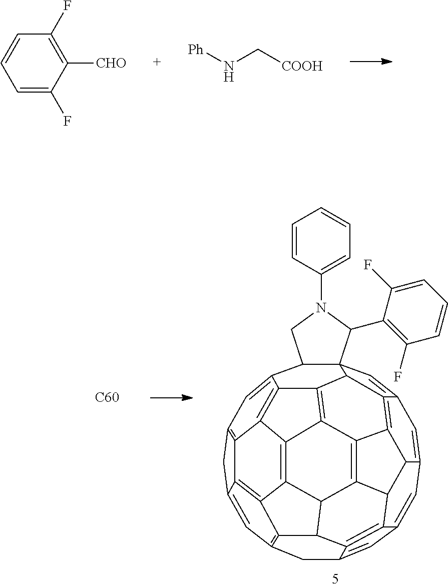 [SMILES: O=Cc1c(F)cccc1F ; O=C(O)CNc1ccccc1 ; "C60" ; Fc1cccc(F)c1C1N(c2ccccc2)CC2c3cc4c5c6c3C3=C7C8=C9C%10C%11=C%12C(=CC%11=CC%11=CC%13=CC21C3C%13=C8C%11%10)Cc1cc(c5c2c1C%12C9c2c67)C4]

2,6-difluorobenzaldehyde (36 mg, 0.25 mmol), N-phenylglycine (76 mg, 0.5 mmol), and C₆₀ fullerene (175 mg, 0.25 mmol) were stirred in 100 mL of toluene at 120° C. for 48 hours.

After cooling, the solvent was distilled off, and the reaction product was separated by silica gel column chromatography (SiO₂, n-hexane:toluene=20:1) to obtain Compound 5 (103 mg, yield: 43%). Compound 5 was further purified by preparative GPC (chloroform).
¹H-NMR (CDCl₃) δ: 5.40 (1H, d-d, J=9.9, 5.9 Hz), 5.66 (1H, d-d, J=9.9, 2.4 Hz), 6.88-7.02 (2H, m), 7.14 (1H, d, J=7.5 Hz), 7.20-7.30 (3H, m), 7.37 (1H, t, J=7.5 Hz).
¹⁹F-NMR (CDCl₃) δ: −105.30-−105.39 (1F), −114.35-−114.45 (1F). MS (FAB) m/z 951 (M+). HRMS calcd for C₇₄H₁₁F₂N 951.0860; found 951.0867.

### Synthesis Example 6

### Synthesis of Compound 6

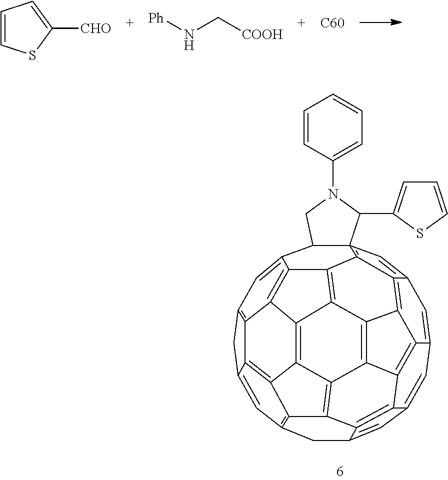

2-thiophenecarbaldehyde (56 mg, 0.5 mmol), N-phenylglycine (76 mg, 0.5 mmol), and C₆₀ fullerene (175 mg, 0.25 mmol) were stirred in 100 mL of toluene at 120° C. for 60 hours. After cooling, the solvent was distilled off, and the reaction product was separated by column chromatography (SiO₂, n-hexane:toluene=10:1 to 2:1) to obtain Compound 6 (113 mg, yield: 49%). Compound 6 was further purified by preparative GPC (chloroform).
¹H-NMR (CDCl₃) δ: 5.11 (1H, d, J=10.1 Hz), 5.60 (1H, d, J=10.1 Hz), 6.60 (1H, s), 6.96-7.02(1H, m), 7.04-7.12(1H, m), 7.22-7.30 (1H, m) 7.32-7.48(5H, m).

### Synthesis Example 7

### Synthesis of Compound 8

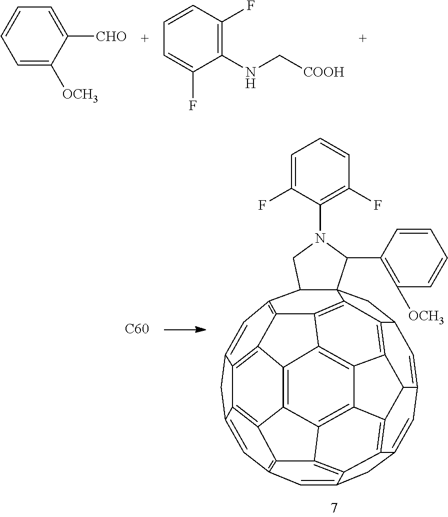 [SMILES: COc1ccccc1C=O ; O=C(O)CNc1c(F)cccc1F ; "C60" ; COc1ccccc1C1N(c2c(F)cccc2F)CC2c3cc4c5c6c(cc7cc8c9c%10c%11c%12c%13c%14c%15c(c%16c3c5c(c%16%13)c3c6c7c9c%123)C21CC%15C=C(C=C%10C8)C%14%11)C4]

C₆₀ fullerene (360 mg, 0.5 mmol), 2-anisaldehyde (136 mg, 1 mmol), and N-(2,6-difluorophenyl)glycine (187 mg, 1 mmol) were stirred in chlorobenzene (100 mL) at 140° C. for 4 days. After cooling, the solvent was distilled off, and the reaction product was separated by silica gel column chromatography (n-hexane:toluene=10:1 to 5:1) to obtain Compound 7 (219 mg, yield: 44.6%). Compound 7 was further purified by preparative GPC (chloroform).
¹H-NMR (CDCl₃) δ: 3.86 (3H, s), 5.20 (1H, d, J=9.1 Hz), 5.34 (1H, d, J=9.1 Hz), 6.88-6.95 (2H, m), 6.99-7.07 (2H, m), 7.16 (1H, s), 7.20-7.29 (2H, m), 7.81 (1H, d, J=7.9 Hz).
¹⁹F-NMR (CDCl₃) δ: −114.80-−114.98 (m).
MS (FAB) m/z 981 (M+). HRMS calcd for C₇₅H₁₃F₂NO 981.0965; found 981.0972.

### Synthesis Example 8

### Synthesis of Compound 8

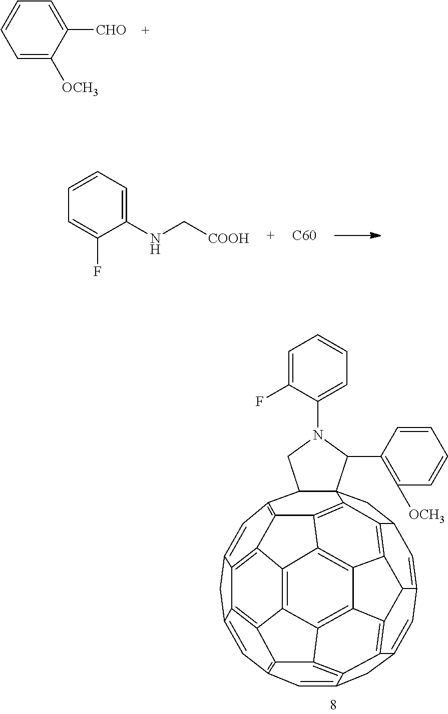 [SMILES: COc1ccccc1C=O ; "C60" ; O=C(O)CNc1ccccc1F ; COc1ccccc1C1N(c2ccccc2F)CC2c3cc4c5c6c(cc7cc8c9c%10c%11c%12c%13c%14c%15c(c%16c3c5c(c%16%13)c3c6c7c9c%123)C21CC%15C=C(C=C%10C8)C%14%11)C4]

C₆₀ fullerene (180 mg, 0.25 mmol), 2-anisaldehyde (68 mg, 0.5 mmol), and N-(2-fluorophenyl)glycine (85 mg, 0.5 mmol) were stirred in chlorobenzene (80 mL) at 130° C. for 4 days. After cooling, the solvent was distilled off, and the reaction product was separated by silica gel column chromatography (n-hexane:toluene=20:1 to 2:1), followed by further purification by preparative GPC (chloroform) to thereby obtain Compound 8 (82.5 mg, yield: 34.2%).
¹H-NMR (CDCl₃) δ: 3.75 (3H, s), 4.71 (1H, d, J=10.3 Hz), 5.65 (1H, d, J=10.3 Hz), 6.68 (1H, s), 6.82-6.93 (2H, m), 7.02-7.29 (5H, m), 7.77 (1H, d-d, J=7.9, 1.6 Hz).
¹⁹F-NMR (CDCl₃) δ: −121.23-−121.34 (m).
MS (FAB) m/z 964 (M+1). HRMS calcd for C₇₅H₁₅FNO 963.1059; found 963.1036.

### Synthesis Example 9

### Synthesis of Compound 9

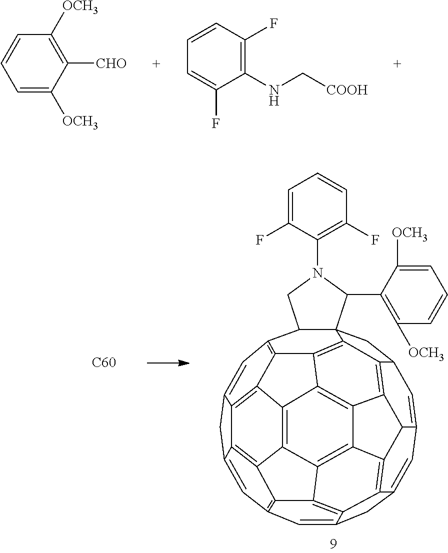 [SMILES: COc1cccc(OC)c1C=O ; O=C(O)CNc1c(F)cccc1F ; "C60" ; COc1cccc(OC)c1C1N(c2c(F)cccc2F)CC2c3cc4c5c6c(cc7cc8c9c%10c%11c%12c%13c%14c%15c(c%16c3c5c(c%16%13)c3c6c7c9c%123)C21CC%15C=C(C=C%10C8)C%14%11)C4]

C₆₀ fullerene (360 mg, 0.5 mmol), 2,6-dimethoxybenzaldehyde (166 mg, 1 mmol), and N-(2,6-difluorophenyl)glycine (187 mg, 1 mmol) were stirred in chlorobenzene (100 mL) at 140° C. for 2 days. After cooling, the solvent was distilled off, and the reaction product was separated by silica gel column chromatography (n-hexane:toluene=10:1 to 5:1) to obtain Compound 9 (205 mg, yield: 40.5%). Compound 9 was further purified by preparative GPC (chloroform).
¹H-NMR (CDCl₃) δ: 3.66 (3H, s), 3.78 (3H, s), 5.04 (1H, d, J=9.1 Hz), 5.52 (1H, d, J=9.1 Hz), 6.38 (1H, d, J=7.7 Hz), 6.55 (1H, d, J=7.7 Hz), 6.86-6.95 (1H, m), 6.97-7.06 (1H, m), 7.11-7.20 (2H, m), 7.38 (1H, S).
¹⁹F-NMR (CDCl₃) δ: −119.23-118.31 (2F, m).
MS (FAB) m/z 1012 (M+1). HRMS calcd for C₇₆H₁₆F₂NO₂ 1012.1149; found 1012.1116.

### Synthesis Example 10

### Synthesis of Compound 10

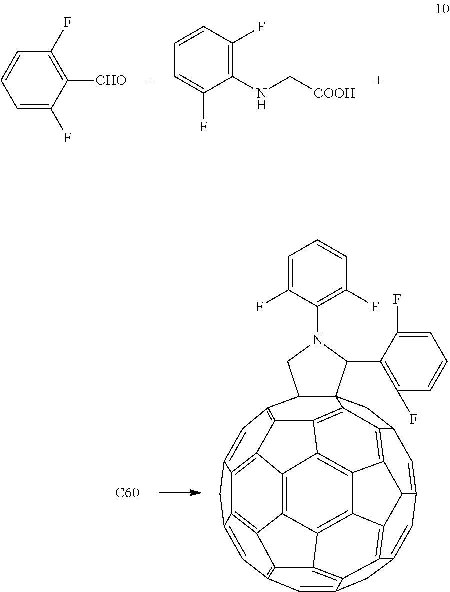 [SMILES: O=Cc1c(F)cccc1F ; O=C(O)CNc1c(F)cccc1F ; "C60" ; Fc1cccc(F)c1C1N(c2c(F)cccc2F)CC2c3cc4c5c6c(cc7cc8c9c%10c%11c%12c%13c%14c%15c(c%16c3c5c(c%16%13)c3c6c7c9c%123)C21CC%15C=C(C=C%10C8)C%14%11)C4]

C₆₀ fullerene (360 mg, 0.5 mmol), 2,6-difluorobenzaldehyde (284 mg, 2 mmol), and N-(2,6-difluorophenyl)glycine (187 mg, 1 mmol) were stirred in chlorobenzene (100 mL) at 140° C. for four days. After cooling, the solvent was distilled off, and the reaction product was separated by silica gel column chromatography (n-hexane:toluene=20:1 to 5:1), followed by further purification by preparative GPC (chloroform) to thereby obtain Compound 10 (108.9 mg, yield: 22.0%).
¹H-NMR (CDCl₃) δ: 5.02 (1H, d-d, J=9.6, 2.4 Hz), 5.41 (1H, d, J=9.6 Hz), 6.76 (2H, t, J=8.8 Hz), 6.91-7.05 (3H, m), 7.06 (1H, d, J=2.4 Hz), 7.08-7.18 (1H, m), 7.18-7.27 (1H, m).
¹⁹F-NMR (CDCl₃) δ: −104.08-104.17 (1F, m), −112.32 (1F, t, J=7.9 Hz), −118.64-118.76 (2F, m).
MS (FAB) m/z 988 (M+1). HRMS calcd for C₇₄H₁₀F₄N 988.0749; found 988.0747.

### Synthesis Example 11

### Synthesis of Compound 11

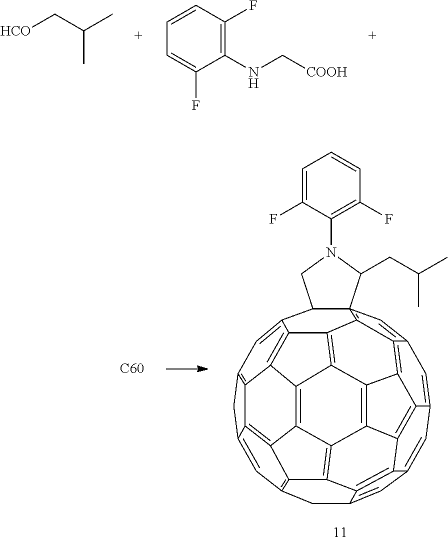

Fullerene C₆₀ (90 mg, 0.12 mmol), isovaleraldehyde (22 mg, 0.25 mmol), and N-(2,6-difluorophenyl)glycine (23 mg, 0.12 mmol) were stirred in chlorobenzene (60 mL) at 130° C. for 3 days. After cooling, the solvent was distilled off, and the reaction product was separated by silica gel column chromatography (n-hexane:toluene=20:1), followed by further purification by preparative GPC (chloroform) to thereby obtain Compound 11 (10.5 mg, yield: 9.0%).
¹H-NMR (CDCl₃-CS₂) δ: 0.95 (3H, d, J=6.4 Hz), 1.03 (3H, d, J=6.4 Hz), 1.88-2.00 (1H, m), 2.36-2.46 (2H, m), 5.12 (1H, d, J=9.6 Hz), 5.19 (1H, d, J=9.6 Hz), 5.40 (1H, d-d, J=6.4, 6.4 Hz), 7.00-7.35 (3H, m).
¹⁹F-NMR (CDCl₃) δ: −117.13-−117.20 (m).
MS (FAB) m/z 932 (M+1). HRMS calcd for C₇₂H₁₄F₂N 931.1173; found 931.1206.

### Synthesis Example 12

### Synthesis of Compound 12

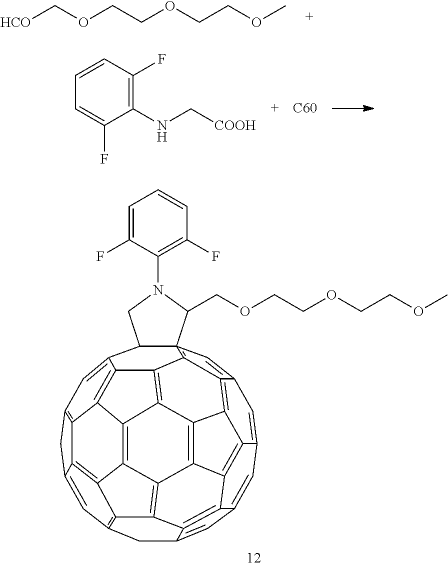

C₆₀ fullerene (180 mg, 0.25 mmol), 2,5,8-trioxadecanal (162 mg, 0.25 mmol), and N-(2,6-difluorophenyl)glycine (46.8 mg, 0.25 mmol) were stirred in chlorobenzene (80 mL) at 135° C. for 4 days. After cooling, the solvent was distilled off, and the reaction product was separated by silica gel column chromatography (toluene:ethyl acetate=50:1), followed by further purification by preparative GPC (chloroform) to thereby obtain Compound 12 (47.9 mg, yield: 19.0%).
¹H-NMR (CDCl₃) δ: 3.33 (3H, s), 3.41-3.72 (6H, m), 4.28 (1H, d-d-d, J=9.9, 5.5, 5.5 Hz), 4.47 (1H, d-d-d, J=9.9, 5.5, 5.5 Hz), 5.12 (1H, d, J=9.6 Hz), 5.19-5.26 (2H, m), 5.30 (1H, d, J=9.6 Hz), 5.54 (1H, d-d, J=6.0, 6.0 Hz), 7.06 (2H, d-d, J=8.5, 8.5 Hz), 7.12-7.25 (1H, m).
F-NMR (CDCl₃) δ: −117.56-−117.65 (m).
MS (FAB) m/z 1009 (M+1). HRMS calcd for C₇₄H₁₉F₂NO₃ 1007.1330; found 1007.1308.

### Synthesis Example 13

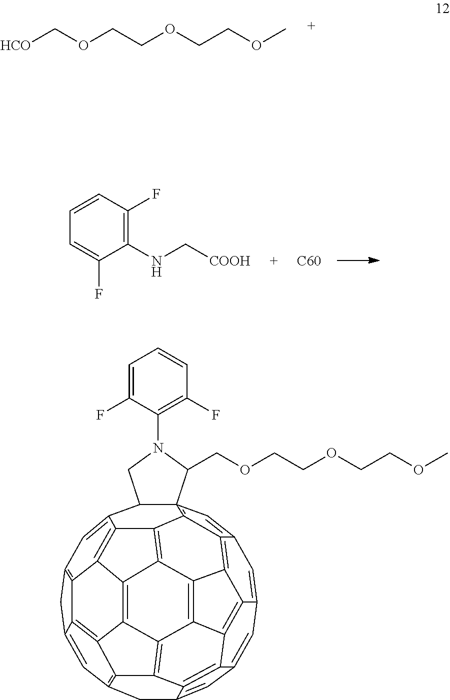

### (1) Synthesis of N-(2,3,5,6-tetrafluorophenyl)glycine

The synthesis was carried out in the manner disclosed in a known document (Brooke, G.M. et al., Tetrahedron, 1971, vol. 27, page 5,653).

A solution of 2,3,5,6-tetrafluoroaniline (2.5 g, 15 mmol) in THF (25 mL) was added dropwise to a solution of sodium hydride (15 mmol) in THF (12 mL) over 1 hour at −30° C. After dropwise addition, the reaction mixture was stirred at room temperature for 1 hour. To this reaction mixture, a solution of ethyl chloroacetate (15 mmol) in THF (12 mL) was added dropwise at room temperature, followed by stirring while heating under reflux for 1 hour. After cooling, the reaction mixture was poured into ice water, and extracted with ether. After dehydration with magnesium sulfate, the extract was concentrated under reduced pressure (yield: 1.8 g). 0.3 g of this reaction product was stirred in 25 mL of a 30% aqueous sodium hydroxide solution under reflux for 3 hours. After cooling, the reaction mixture was adjusted to a pH of 3 with concentrated hydrochloric acid, and extracted with ethyl acetate. The organic phase was washed with water, dehydrated with magnesium sulfate, and concentrated under reduced pressure (yield: 1.2 g).
¹H-NMR (CD₃OD) δ: 4.00-4.14 (2H, m), 6.48-6.61 (1H, m).
¹⁹F-NMR (CD₃OD) δ: −-144.11-−144.25 (2F, m), −163.14-−163.28 (2F, m).

### (2) Synthesis of Compound 13

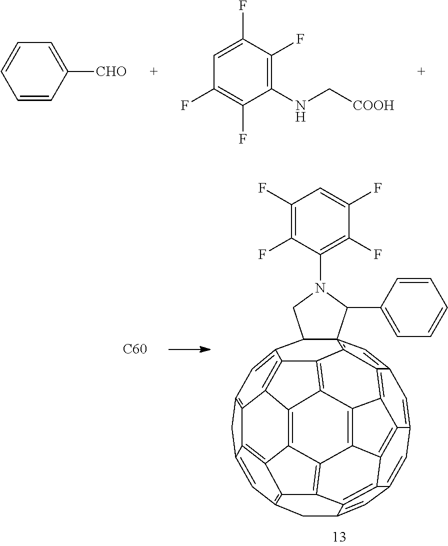

C₆₀ fullerene (180 mg, 0.25 mmol), benzaldehyde (1.06 g, 10 mmol), and N-(2,3,5,6-tetrafluorophenyl)glycine (45 mg, 0.2 mmol) were stirred in chlorobenzene (100 mL) at 145° C. for 2 days. After cooling, the solvent was distilled off. The target product was confirmed by peaks at 4.60 (1H, d, J=10.0 Hz), 5.66 (1H, D, J=10.0 Hz), and 5.84 (1H, s) (6, (ppm)) in ¹H-NMR (CDCl₃), and peaks at −138.70-−138.90 (2F, m), and −150.20-−150.35 (2F, m) (6 (ppm)) in ¹⁹F-NMR (CDCl₃).

### Compound 14: Synthesis of Compound 14

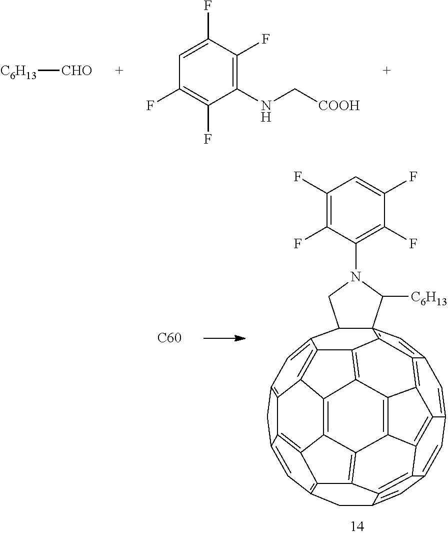

C₆₀ fullerene (180 mg, 0.25 mmol), pentanal (1 mL), and N-(2,3,5,6-tetrafluorophenyl)glycine (45 mg, 0.2 mmol) were stirred in chlorobenzene (100 mL) at 145° C. for 4 days. After cooling, the solvent was distilled off, and the reaction product was separated by silica gel column chromatography (hexane:toluene=20:1). The target product was obtained (11.8 mg, yield: 4.8%).
¹H-NMR (CDCl₃) δ: 0.85 (3H, d, J=6.4 Hz), 1.20-1.70 (8H, m), 2.40-2.60 (2H, m), 5.11 (1H, d, J=9.6 Hz), 5.37 (1H, d, J=9.6 Hz), 5.50 (1H, d-d, J=6.4, 6.4 Hz), 6.85-7.00 (1H, m).
¹⁹-NMR (CDCl₃) δ: −139.00-−139.20 (2F, m), −146.60-−146.80 (2F, m).

### Synthesis of Compound 15

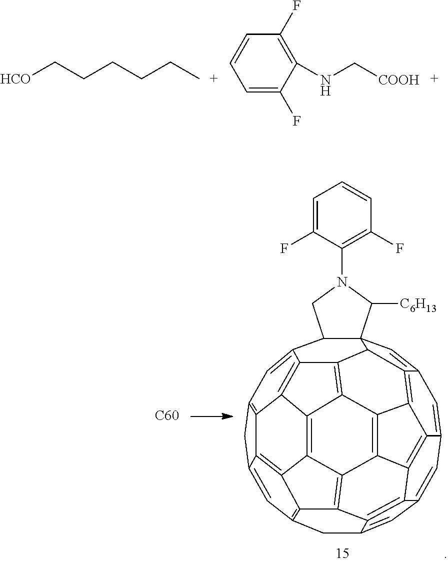

C₆₀ fullerene (360 mg, 0.50 mmol), pentanal (1 mL), and N-(2,6-difluorophenyl)glycine (94 mg, 0.50 mmol) were stirred in chlorobenzene (150 mL) at 130° C. for 4 days. After cooling, the solvent was distilled off, and the reaction product was separated by silica gel column chromatography (n-hexane:toluene=50:1), followed by further purification by preparative HPLC (column used: Cosmosil Buckyprep (20Φ×250 mm); Nacalai Tesque, Inc.; solvent: toluene) to thereby obtain Compound 15 (35.5 mg, yield: 7.4%).
¹H-NMR (CDCl₃) δ: 0.81 (3H, d, J=6.9 Hz), 1.10-1.75 (8H, m), 2.25-2.36 (1H, m), 2.44-2.55 (1H, m), 5.08 (1H, d, J=9.8 Hz), 5.18 (1H, d, J=9.8 Hz), 5.50 (1H, d-d, J=6.9, 5.9 Hz), 7.08 (2H, d-d, J=8.7, 8.7 Hz), 7.15-7.28 (1H, m).
¹⁹F-NMR (CDCl₃) δ: −117.76 (2F,d-d, J=7.5, 7.0 Hz).

### Test Example 1

Solar cell was produced in accordance with the following procedure using fullerene derivative obtained in

Synthesis Example 4 as an n-type semiconductor material, and the performance was evaluated.

The following materials were used: PTB7 as an organic p-type semiconductor material, PFN(poly[9,9-bis(3′-(N,N-dimethylamino)propyl-2,7-fluorene)-alt-2,7-(9,9-dioctylfluorene)]) and MoO₃ (molybdenum oxide) as charge transport layer materials, and ITO (indium tin oxide) (negative electrode) and aluminium (positive electrode) as electrodes.

### (1) Preparation of Solar Cell for Testing

Solar cells for testing were prepared in accordance with the following procedure.

### 1) Pretreatment on Substrate

An ITO patterning glass plate (manufactured by Sanyo Vacuum Industries Co.,Ltd.) was placed in a plasma cleaner (Harrick plasma, PDC-32G), and the surface of the substrate was washed with generated plasma while oxygen gas was being introduced for 10 minutes.

### 2) Preparation of PFN Thin Film (Charge Transport Layer on Negative Electrode Side)

A PFN thin film was formed using a PFN methanol solution (2% w/v) on the pretreated ITO glass plate by using an ABLE/ASS-301 spin-coating-film-forming apparatus. The formed PFN thin film had a thickness of about 10 nm.

### 3) Preparation of Organic Semiconductor Film (Organic Power-Generating Layer)

With the substrate placed in a glove box, the PFN thin film was spin-coated with a solution containing PTB7 which were dissolved in chlorobenzene beforehand and the fullerene derivative, and diiodooctane (3% v/v relative to chlorobenzene), using a MIKASA/MS-100 spin-coating film-forming apparatus at 1,000 rpm for 2 minutes to thereby obtain an organic semiconductor thin film (organic power-generating layer) of about 90 to 110 nm.

### 4) Vacuum Deposition of Charge Transport Layer on Positive Electrode Side and Vacuum Deposition of Metal Electrode

The above-prepared laminate was placed on a mask inside a compact high vacuum evaporator (Eiko Co., Ltd., VX-20). An MoO₃ layer (10 nm) as a charge transport layer on the positive electrode side and an aluminium layer (80 nm) as a metal electrode were deposited thereon in series using the high vacuum evaporator.

### (2) Current Measurement by Pseudo Solar Light Irradiation

Current measurement using pseudo solar light irradiation was conducted by using SourceMeter (Keithley, Model 2400), current-voltage measuring software and a solar simulator (San-Ei Electric Co., Ltd., XES-3015).

The solar cells for testing produced in section (1) were irradiated with a given amount of pseudo solar light, and the generated current and voltage were measured. Energy conversion efficiency was then determined by the following equation.

Table 1 shows the measurement results of short-circuit current, open voltage, fill factor (FF), and conversion efficiency. The conversion efficiency is a value determined by the following equation.

Conversion efficiency 11 (%)=FF (V_{oc}×J_{sc}/Pᵢₙ)×100

FF: Fill Factor, V_{oc}: Open voltage, J_{sc}: Short-circuit Current,
Pᵢₙ: Intensity of Incident Light (Density)

TABLE 1

| | Short-circuit | Open | | Conversion |
|---|---|---|---|---|
| | Current | Voltage | | Efficiency |
| | (mA/cm²) | (V) | FF | (%) |
| Test Example 1 | 12.17 | 0.77 | 0.61 | 5.71 |

### Test Example 2

Comparative Compound 1 of the below-described structural formula, and the fullerene derivatives obtained in Synthesis Examples 1 to 10 were used as n-type semiconductor materials to thereby prepare solar cells having the same cell structure as that of Test Example 1 in accordance with the following procedure. The performance of each fullerene derivative was then evaluated. Each of the fullerene derivatives was subjected to column separation, and further purified by HPLC (used column: Cosmosil Buckyprep (20Φ×250 mm); Nacalai Tesque, Inc.; solvent:toluene) for use.

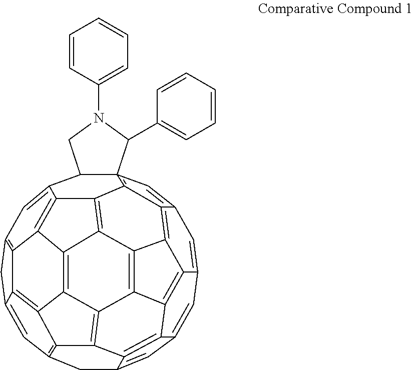

Table 2 shows the results.

TABLE 2

| | p-type | Short-circuit | Open | | Conversion |
|---|---|---|---|---|---|
| Fullerene | Semi- | Current | Voltage | | Efficiency |
| Derivative | conductor | (mA/cm²) | (V) | FF | (%) |
| Comparative | PTB7 | 14.54 | 0.74 | 0.66 | 7.16 |
| Compound 1 | | | | | |
| Compound 1 | PTB7 | 14.33 | 0.73 | 0.65 | 6.84 |
| Compound 3 | PTB7 | 14.84 | 0.78 | 0.61 | 7.09 |
| Compound 4 | PTB7 | 14.47 | 0.76 | 0.64 | 7.12 |
| Compound 5 | PTB7 | 14.85 | 0.76 | 0.61 | 6.88 |
| Compound 7 | PTB7 | 14.76 | 0.80 | 0.55 | 6.45 |
| Compound 8 | PTB7 | 14.76 | 0.77 | 0.60 | 6.76 |
| Compound 9 | PTB7 | 13.88 | 0.81 | 0.55 | 6.10 |
| Compound 10 | PTB7 | 13.72 | 0.79 | 0.60 | 6.53 |
| Comparative | PTB7 | 14.21 | 0.76 | 0.67 | 7.27 |
| Compound 2 | | | | | |
| Compound 15 | PTB7 | 13.55 | 0.82 | 0.47 | 5.21 |

### Test Example 3

Comparative compound 2 of the below-described structural formula and Compound 15 were used as n-type semiconductor materials to thereby produce solar cells having the same cell structures as those of Test Examples 1 and 2 in accordance with the following procedure. The performance of each fullerene derivative was then evaluated. Comparative Compounds 1 and 2 were synthesized in accordance with Patent Document 3.

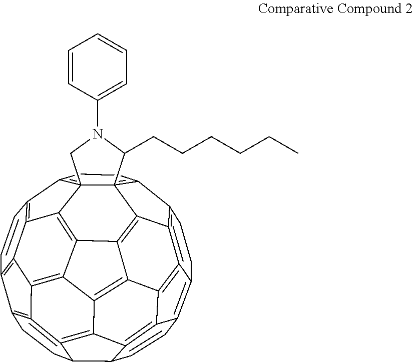

Compared with Comparative Examples 1 and 2 whose phenyl groups are not substituted (Comparative Compounds 1 and 2), the solar cells comprising the compounds according to the present invention all show improved open voltage. The open voltage also improves in accordance with the number of substituents.

This trend is more noticeable with the phenyl group attached to the pyrrolidine ring at position 1 (nitrogen) than the phenyl group attached to the pyrrolidine ring at position 2. Although there are some documents mentioning substituents of phenyl contained in a fullerene derivative and open voltage (below-listed documents 1) to 4)), there have been no such findings concerning phenyl attached to the nitrogen of a pyrrolidine-containing derivative. Further, there have been no previous examples of a fullerene derivative whose pyrrolidine ring is substituted with the above-described substituents at both positions 1 and 2.

### REFERENCE

1) Ito et al., Journal of Materials Chemistry, 2010, vol. 20, page 9,226 (Non-patent Document 1)
2) Hummelen et al., Organic Letters, 2007, vol. 9, page 551
3) Troshin et al., Advanced Functional Materials, 2009, vol. 19, page 779
4) JP2011-181719A

1. A fullerene derivative represented by formula (1): 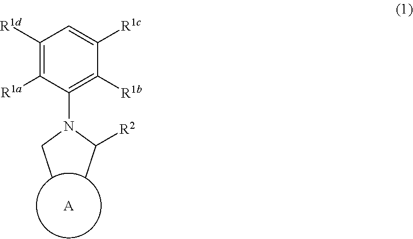
wherein
R^{1a} and R^{1b} are the same or different, and each represents a hydrogen atom or a fluorine atom;
R^{1c} and R^{1d} are the same or different, and each represents a hydrogen atom, a fluorine atom, alkyl optionally substituted with at least one fluorine atom, alkoxy optionally substituted with at least one fluorine atom, ester, or cyano;
R² represents
(1) phenyl optionally substituted with at least one substituent selected from the group consisting of fluorine, alkyl, alkoxy, ester, and cyano,
(2) a 5-membered heteroaryl group optionally substituted with 1 to 3 methyl groups, or (3) alkyl, alkoxy, ether, acyl, ester, or cyano; and
ring A represents a fullerene ring;
with the proviso that when R^{1a}, R^{1b}, R^{1c}, and R^{1d} are each a hydrogen atom, R² represents phenyl substituted with 1 or 2 fluorine atoms or a 5-membered heteroaryl group optionally substituted with 1 to 3 methyl groups.

2. The fullerene derivative according to claim 1, wherein
R^{1a} and R^{1b} are the same or different, and each represents a hydrogen atom or a fluorine atom;
at least one of R^{1a} and R^{1b} is a fluorine atom; and
R² is a group represented by the following formula: 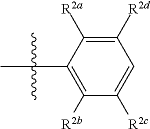
wherein,
R²a and R²b are the same or different, and each represents a hydrogen atom, a fluorine atom, alkyl, or alkoxy; and
R²c and R²d are the same or different, and each represents a hydrogen atom, a fluorine atom, alkyl, alkoxy, ester, or cyano.

3. The fullerene derivative according to claim 2, wherein R^{1a} and R^{1b} are the same or different, and each represents a hydrogen atom or a fluorine atom;
at least one of R^{1a} and R^{1b} is a fluorine atom;
R^{1c} and R^{1d} are the same or different, and each represents a hydrogen atom or a fluorine atom;
R²a and R²b are the same or different, and each represents a hydrogen atom, a fluorine atom, alkyl, or alkoxy; and
R²c and R²d each represents a hydrogen atom.

4. The fullerene derivative according to claim 1, wherein the ring A is C₆₀ fullerene or C₇₀ fullerene.

5. An n-type semiconductor material consisting of the fullerene derivative according to claim 1.

6. The n-type semiconductor material according to claim 5, which is for use in an organic thin-film solar cell.

7. An organic power-generating layer comprising the n-type semiconductor material according to claim 6.

8. A photoelectric conversion element comprising the organic power-generating layer according to claim 7.

9. The photoelectric conversion element according to claim 8, which is an organic thin-film solar cell.
